(19) United States
(12) Patent Application Publication
(10) Pub. No.: US 2025/0332116 A1
(43) Pub. Date: Oct. 30, 2025

(54) CARBON-BASED ANTI VIRAL NANOPARTICLES

(71) Applicants: Plinio INNOCENZI, Alghero (IT); Luigi STAGI, Siddi (IT); Luca MALFATTI, Alghero (IT); PORTO CONTE RICERCHE SOCIETA A RESPONSABILITA LIMITATA, Alghero (IT)

(72) Inventors: Plinio INNOCENZI, Alghero (IT); Luigi STAGI, Siddi (IT); Luca MALFATTI, Alghero (IT)

(21) Appl. No.: 18/555,389

(22) PCT Filed: Apr. 12, 2022
(86) PCT No.: PCT/IB2022/053416

§ 371 Date: Oct. 13, 2023

(30) Foreign Application Priority Data
Apr. 14, 2021 (IT) 102021000009410

(51) Int. Cl. A61K 9/51; A61L 31/10; A61P 31/14

(52) U.S. Cl. CPC A61K 9/5146; A61L 31/10; A61P 31/14

(57) ABSTRACT

The present invention relates to new carbon-based antiviral nanoparticles, compositions comprising said nanoparticles, materials and devices coated with said nanoparticles, said nanoparticles for medical use, methods for the preparation of said nanoparticles, compositions, materials and devices.

### FIELD OF THE INVENTION

The present invention relates to new carbon-based antiviral nanoparticles, compositions comprising said nanoparticles, materials and devices coated with said nanoparticles, said nanoparticles for medical use, methods for the preparation of said nanoparticles, compositions, materials and devices.

### STATE OF THE ART

Infectious diseases account for 20% of global mortality and viruses are responsible for about one third of these deaths. Among the most common causes of death are lower respiratory tract infections and human immunodeficiency viruses (HIV) and represent a major expense for national health systems. In addition to these well-known viruses, new ones emerge every year (e.g., the more recent SARS-CoV-2). The best approach to fight viral infections is represented by vaccines, but there is a limited number of effective vaccines and they are often not available in all parts of the world.

A key role in a successful response in this fight is represented by antiviral drugs. However, said drugs are often limited by the virus specificity, and are in many cases ineffective with the emergence of new and unknown viral strains. In the latter case, the only possibilities of counteracting the development of the virus lies in the treatment of symptoms and the application of protocols to contain its spread, i.e., the use of protection and isolation devices as well as social distancing. Currently, many therapies are based on the use of small molecules (e.g., peptidomimetics), proteins that stimulate the immune response (e.g., interferon), and oligonucleotides. Virucidal substances are able to interfere in the early stages of the virus replication cycle. Their efficacy is based on their ability to form bonds with viral particles, preventing them from interacting with healthy cells. However, in many cases these substances have the disadvantage of separating from the viral particles after dilution, leaving them unaltered and free to attack the cell. Virucidal substances, on the other hand, have the ability to irreversibly deactivate viral particles. These substances include detergents, acids, polymers and nanoparticles.

Despite their extraordinary effectiveness, they are highly toxic, making them unusable in most cases. It is exceptionally important to be able to synthesise substances in which the low or non-toxic properties of virucidal systems and the effectiveness of virucidal substances coincide.

In recent years, the lively research on carbon-based nanomaterials has revealed that some systems have properties of interest for nanomedical purposes. These include carbon dots (C-dots). C-dots have been extensively investigated for their excellent properties in photonics, optoelectronics and photocatalysis. In addition, C-dots appear to play a decisive role both as luminescent markers and as active systems against viral agents. One of the key features of C-dots is the ease of implementation and low cost, which allows the realisation of systems controlled in chemical composition and size, typically below 10 nm. In a common synthesis, a carbon-based precursor undergoes controlled thermal decomposition under wet or dry conditions. The product can be engineered by reacting nitrogen sources with carbon precursors or by performing post-synthesis functionalisation. Studies performed on Vero cells, MOLT-4 A549, MARC-145, PK-15 and many others, confirm the excellent biocompatibility of C-dots, being non-cytotoxic even at high concentrations of several hundred μg mL⁻¹. C-dots with controlled morphology, derived from various monomers, have been shown to be effective against HSV-1, HIV-1, PRRSV, PRV, HCoV-229E and flaviviruses (JEV). Although there are a number of encouraging results, the mechanism of virus inhibition by C-dots is controversial and still debated. In particular, it is believed that C-dots with specific functional groups on the surface may interact with the cell membrane, preventing virions from attacking the system. More recently, on the other hand, benzoxazine-derived C-dots have been shown to be effective in hindering viral activity by interacting directly with virions, constituting the first true dots system capable of actively opposing viral particles and displaying broad-spectrum antiviral activity.

The worldwide emergency created by the Covid-19 outbreak has found the world unprepared in all fields i.e. prevention, therapy and vaccination.

Covid-19 pandemic has mobilized the research to explore new solutions for vaccines and therapeutic agents against SARS-CoV-2. An important challenge to face is the virus capability of fast genetic mutations, which could hamper developing effective vaccines for all the possible strains as well as the high R₀ of the virus. For these reasons, it is crucial to make available broad range active antiviral systems. Innovative methods based on nanomaterials have risen the attention to suppressing the virus' spread with a special emphasis to sanitize contaminated surfaces. Fewer efforts have been dedicated to investigating the application of nanomaterials and nanostructures as antiviral systems to be potentially applied in vivo. The first requirement nanomaterials have to satisfy to be used as in vivo antibacterial or antiviral systems is the lack of cytotoxicity. For this reason, carbon nanomaterials are one of the main candidates to be used as antiviral systems because several independent studies have demonstrated low cytotoxicity in vitro in several cell lines. Within the family of carbon nanomaterials, fullerenes, graphene, graphene oxide and carbon dots have shown antiviral properties and have been tested with different types of viruses, including coronaviruses (Innocenzi et al Carbon-Based Antiviral Nanomaterials: Graphene, C-Dots, and Fullerenes. A Perspective. Chem. Sci. 11, 6606-6622 (2020)). Fullerenes exert an antiviral activity mainly under UV illumination via the production of reactive oxygen species, while the interaction of graphene and graphene oxide with viruses are quite complex, and the antiviral properties are associated with wrapping, trapping or physical disruption by the sharp-edged structure. However, the graphene sheets' dimensions and the difficulty in achieving precise control of surface functionalization represent severe limitations.

Even if the research is still at the initial stage, carbon dots are suggested among the most promising candidates as antiviral carbon-based nanomaterials, and recent experiments have shown encouraging results. In particular, carbon nanoparticles are potentially attractive as virucidal systems because they can interfere with the virus capability to enter the cells. In vitro experiments have shown that functional carbon dots protect the host cells from infection by HCoV-229E coronavirus and HIV-1. Other potential advantages are low cytotoxicity, the reduced synthesis cost, the potential broad-spectrum application to different viruses, the possibility to be handled without following severe protocols. On the other hand, the lack of systematic research in the field still requires a long-term experimental assessment to evaluate the effective extension in vivo of virucidal carbon-based nanomaterials.

### SUMMARY OF THE INVENTION

The present inventors have tested the antiviral properties of various carbon nanomaterials and have found that an alternative route to carbon dots and carbon quantum dots, which are characterized in general by a graphitic core, as potential antiviral systems, is represented by specific carbon nano-polymeric materials. Carbon nano-polymeric materials are characterized by a polymeric and flexible structure whose dimension is generally very similar or shortly larger than a virus and can interfere with its infection cycle. In particular, the inventors found that materials obtained by thermal polymerisation of a positively charged amino acid in the presence of boric acid were highly effective against SARS-CoV-2 virus infection in a Biosafety level 3 (BSL3) facility. The inventors surprisingly found not only the dimension, but also the surface charge, expressed as zeta potential, of the nanomaterial was an essential feature against SARS-CoV-2 viral infection and that the presence of boric acid during the thermal polymerisation was essential in order to obtain a nano-material with the desired zeta potential (see comparative data below).

In fact, as shown in the comparative data provided in the present description, the thermal polymerization reaction of the same positively charged amino acid, carried out in the presence or in the absence of boric acid, provided two different nanomaterials, said difference being at least in the dimension and in the zeta potential of the nanomaterials obtained. Surprisingly, the two nanomaterials, obtained from the thermal polymerisation of the same positively charged amino acid proved to have an extremely different antiviral effect. In fact, the nanomaterial obtained by thermal polymerization of a positively charged amino acid in the presence of boric acid showed a strong antiviral property as opposed to the nanomaterial obtained by thermal polymerization of the same amino acid in the absence of boric acid.

The experimental data obtained demonstrate the high effectiveness of the nanomaterial of the invention as well as the non-cytotoxicity of the same.

Additionally, the nanomaterial disclosed and claimed herein, is dispersible in water and DMSO.

Therefore, the nanomaterial of the invention, is characterised by a low cost for production, a low cytotoxicity, a high antiviral effect and a high dispersibility in water. The skilled reader will appreciate that all the above-mentioned features are highly desirable in a product for medical use.

The present invention therefore relates to:
A process for the preparation of a nanoparticle comprising a positively charged amino acid hyperbranched nanopolymer, wherein said particle has a three-dimensional dimension from 100 to 400 nm in each dimension and a zeta potential of +10-30 mV and wherein said positively charged amino acid is subjected to thermal polymerisation in the presence of boric acid.
A nanoparticle comprising a positively charged amino acid branched nanopolymer, wherein said particle has a three-dimensional dimension from 100 to 400 nm in each dimension and a zeta potential of +10-30 mV obtainable by said process;
A composition comprising said nanoparticle;
A product coated with said nanoparticle or said composition;
Said nanoparticle for use in a medical treatment;
A medical treatment wherein said nanoparticle or said composition is administered in a therapeutically effective dosage.

### Glossary

In the present description and figures, the term “lysine-only nanopolymer” or “lysine-only nanomaterial” refers to the thermal polymerisation product of the sole dry starting material L-lysine obtainable as described in example 7.

In the present description and figures, the term “lysine-B nanomaterial” refers to the thermal polymerisation product of the starting materials L-lysine and Boric Acid according to the process disclosed and claimed in the description, also defined, herein, as “nanoparticle comprising a L-lysine branched nanopolymer wherein said particle has a three-dimensional dimension from 100 to 400 nm in each dimension and a zeta potential of +10-30 mV obtainable by thermal polymerisation in the presence of boric acid” as disclosed, claimed and described in the present application (e.g. the nanoparticle obtainable as disclosed in example 7 by a L-Lysine and Boric Acid mixture)

The term positively charged amino acid according to the present description has the meaning commonly intended in the state of the art and refers to one of L-Lysine, L-Arginine, L-Histidine, D-Lysine, D-Arginine, D-Histidine.

The term lysine in any part of the present description, drawings and claims always refers to L-lysine also when not specified.

The term “hyperbranched” when referred to the positively charged amino acid branched nanopolymer described and claimed herein, has the meaning commonly used in the state of the art, and therefore refers to highly branched positively charged amino acid macromolecules that are prepared through a single-step polymerization process, in the state of the art, it is known that hyperbranched polymers are built up from dendritic, linear, and terminal units. They can be synthesized via three routes:
1. single-step polymerisation of monomers as the nanopolymers of the present invention,
2. Self-condensing vinyl polymerization of monomers,
3. Multibranching ring opening polymerization of latent monomers.

The degree of branching (DB) and the average number of branches (ANB) can be calculated using the integrals of the different structural units in the ¹H spectra using the formulas according to Scholl, M., et al Controlling Polymer Architecture in the Thermal Hyperbranched Polymerization of L-Lysine. Macromolecules 40, 5726-5734 (2007):

$$DB = \frac{D + T}{D + L + T} = \frac{D + T}{D + {L}_{α} + {L}_{ε} + T} \qquad ( 1 )$$

$$and$$

$$ANB = \frac{D}{D + L} \qquad ( 2 )$$

with D the dendritic units, T the terminal structural units, Lₐ the N^{a}-linked linear units, Le the N^{e}-linked linear units.
branching

Thermal polymerisation according to the present description in the present description and claims has the meaning commonly intended in the state of the art and refers to a reaction in which monomers are converted to polymers by thermal energy.

### DETAILED DESCRIPTION OF THE DRAWINGS

a) In situ FTIR spectra in the 1800-1475 cm⁻¹ range during thermal treatment of the Lysine-H₃BO₃ mixture (1:1 molar ratio) from 25 to 250° C. The absorbance is reported in false colour scale.
b) FTIR spectra of pure lysine and lysine-B nanomaterial in the 4000-400 cm⁻¹ range (gray and black line, respectively).
c) DSC analysis of the Lysine-H₃BO₃ mixture indicated as HPNs in the figure (1:1 molar ratio) from 25 to 500° C. (gray line). The black line shows the thermal ramp with a 20 min dwell at the amidation temperature.
d) ¹H NMR spectra of lysine (top) and hyperbranched polylysine nanopolymers (middle). Attribution of the chemical shifts (bottom) has been done following the data published in: Scholl, M., Nguyen, T. Q., Bruchmann, B., Klok, H.-A. The Thermal Polymerization of Amino Acids Revisited; Synthesis and Structural Characterization of Hyperbranched Polymers from L-Lysine. J. Polymer Sci.: Part A: Polymer Chem., 45, 5494-5508 (2007). The ¹H NMR data are consistent with the formation of a hyperbranched polylysine structure via thermal induced amidation reactions, in accordance with FTIR and DSC data.
2.67 ppm e-CH₂ group in a a-linear and terminal structural unit
3.19 ppm e-CH₂ group next to an amide bond (dendritic and e-linear structural unit)
3.25 ppm a-CH protons of the e-linear structural units
3.33 ppm a-CH protons of the terminal structural units
4.02 ppm a-CH protons of the a-linear structural units
4.24 ppm a-CH protons of the dendritic protons
e) Scheme for the formation of a hyperbranched polylysine via thermal polymerization.

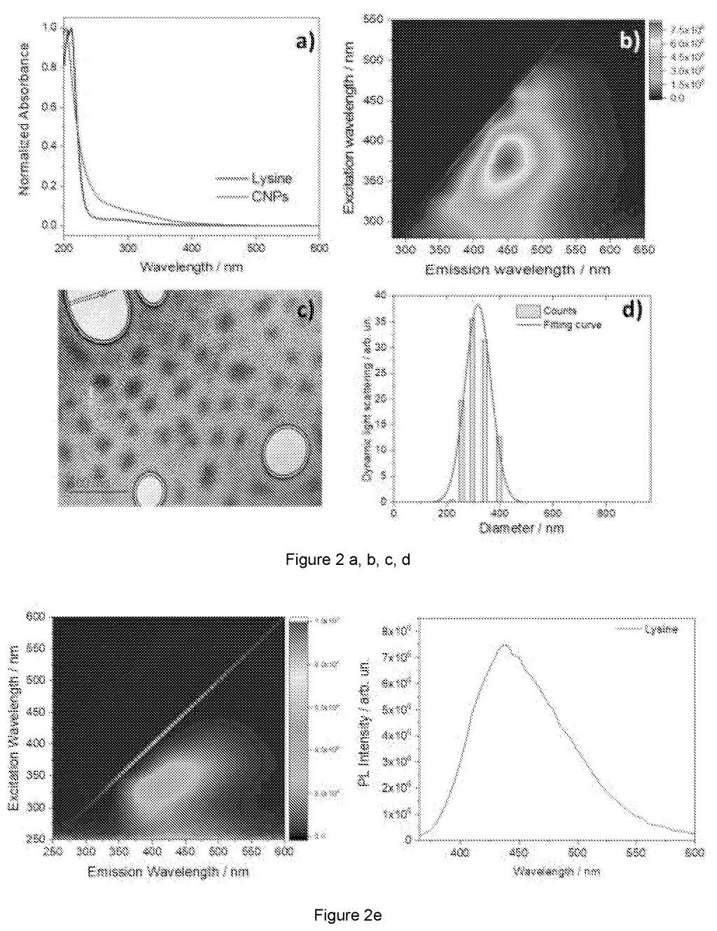

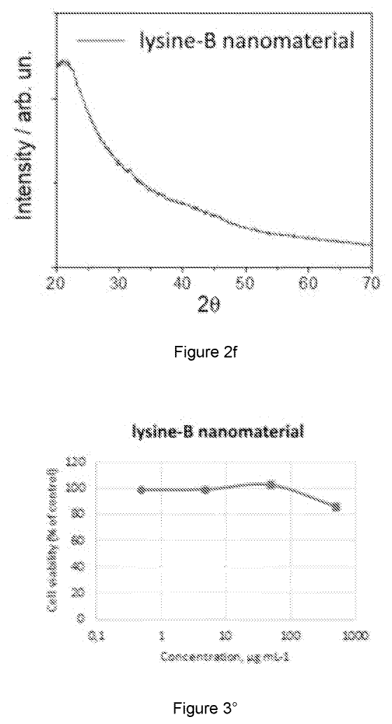

FIG. 2.
a) UV-Vis absorption spectra of lysine (black line) (0.01 M) and lysine-B nanomaterial (red line) in water (0.25 mg mL⁻¹).
b) Excitation (y axis)—Emission (x axis)—Intensity (false colour scale) fluorescence map of the lysine-B nanomaterial in water (0.25 mg mL⁻¹).
c) Representative bright-field TEM image of lysine-B nanomaterial.
d) Dynamic light scattering of the lysine-B nanomaterial in water (0.25 mg mL⁻¹).
e) Three-dimensional fluorescence graph [excitation (y)—emission (x)—intensity (z)] of lysine in water (left). Emission spectrum of lysine upon excitation at 350 nm (right).
f) X-ray diffraction analysis of the lysine-B nanomaterial sample. The diffraction pattern supports the amorphous nature of the lysine-B nanomaterial

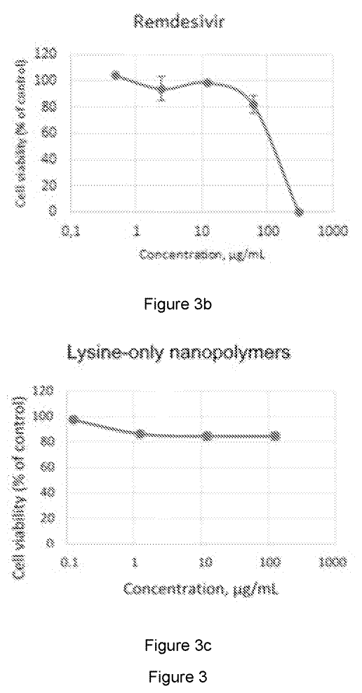

FIG. 3.
a. Cytotoxicity of lysine-B nanomaterial
b. Cytotoxicity of reference drug Remdesivir
c. Cytotoxicity of lysine-only nanopolymers.

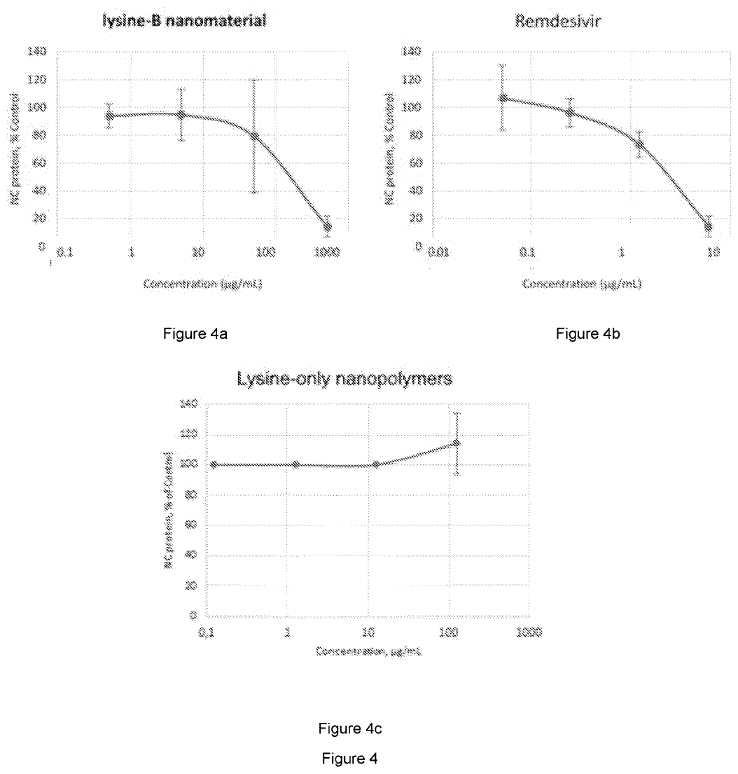

FIG. 4.
Antiviral activity:
a. Effect of lysine-B nanomaterial on SARS-CoV-2 replication
b. Effect of remdesivir drug on SARS-CoV-2 replication
c. Effect of lysine-only nanopolymers on SARS-CoV-2 replication

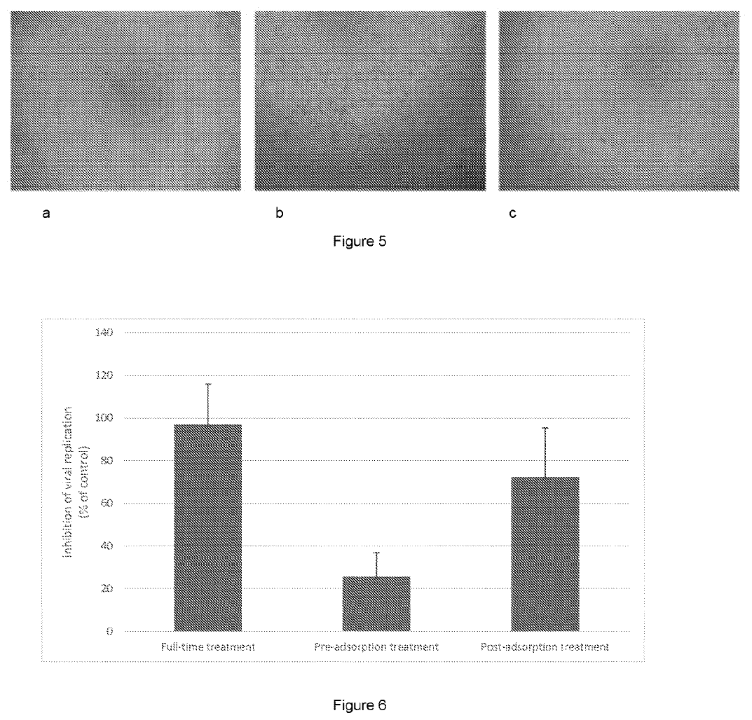

FIG. 5. Optical images of Vero E6 monolayer:
a) uninfected control,
b) infected with SARS-CoV-2,
c) infected with SARS-CoV-2 and treated with lysine-B nanomaterial at 250 μg mL⁻¹.

FIG. 6. Time-of-addition experiments. Inhibition of viral replication (i.e., production of viral nucleocapsid protein detected through ELISA 72 hours post infection) is shown for full-time treatment, pre-adsorption treatment and post-adsorption treatment.

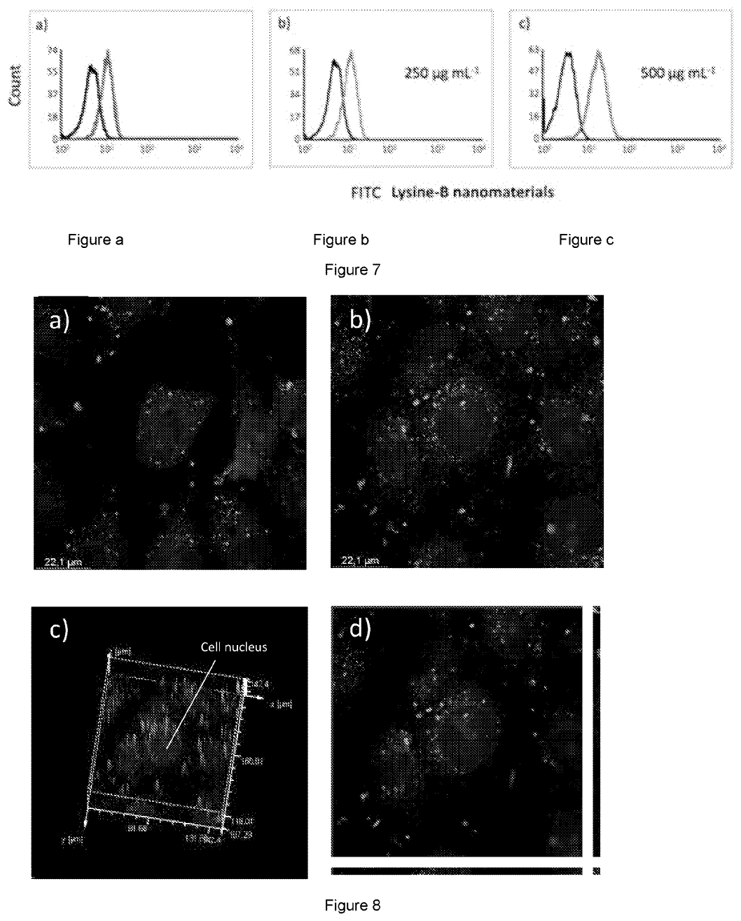

FIG. 7.
(a) Flow cytometry histograms for cells pulsed with 250 μg mL⁻¹ of the lysine-B nanomaterial and analyzed after 24 hours (gray), control cells are shown in black. Flow cytometry histograms for cells treated with 250 μg mL⁻¹
(b) and 500 μg mL⁻¹
(c) of the nanomaterial for 24 hours; treated cells are shown in red compared to control cells in black.

FIG. 8. Confocal microscope images of control:
a) and b) cells pulsed with nanomaterial at 250 μg mL⁻¹ for 24 hours;
c) 3D projection of fluorescence distribution within the cell;
d) 3D rendering through side projections of confocal Z-stack confirming nanoparticles (green spots). The slides were analysed by Leica SP5 confocal microscope.

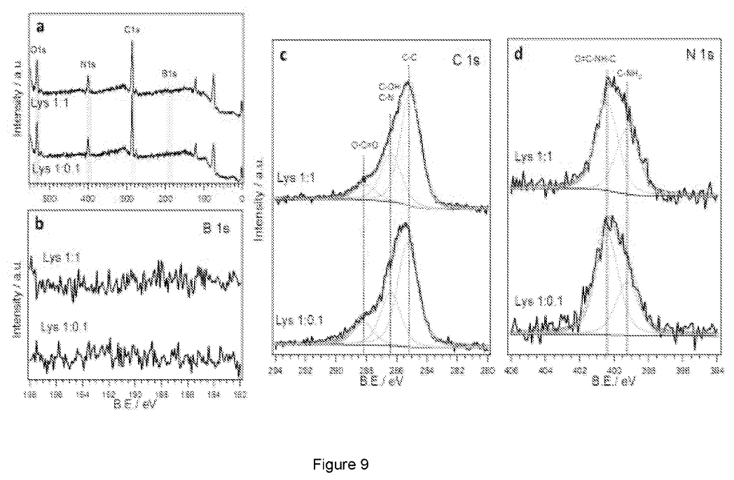

FIG. 9. XPS analysis. Survey (a) and high resolution B 1s (b), C 1s (c), and N 1s (d) XPS regions. The separation into chemical shifted components is included in (c) and (d).

### DETAILED DESCRIPTION OF THE INVENTION

The present invention hence refers to a process that provides a new lysine based nanomaterial, herein also referred to as lysine-B nanomaterial, said nanomaterial having high antiviral activity, low cytotoxicity, low production costs, high solubility in hydrophilic solvents including water. The results provided in the figures and in the experimental data demonstrate the surprising antiviral properties of the nanomaterial obtainable by thermal polymerisation in the presence of boric acid of a positively charged amino acid. In particular, the data provided herein, demonstrate that, surprisingly, the presence of boric acid during the thermal polymerisation process of a positively charged amino acid confers to the nanomaterial obtainable by said polymerisation a strong antiviral property that is totally absent from the nanomaterial obtainable from the same positively charged amino acid (FIG. 4).

An object of the invention is therefore a process for the preparation of a nanoparticle comprising a positively charged amino acid branched nanopolymer,
wherein said nanoparticle has a three-dimensional dimension from 100 to 400 nm in each dimension and a zeta potential of +10-30 mV, and wherein said positively charged amino acid is subjected to thermal polymerisation in the presence of boric acid.

According to the present invention, the surface charge has been assessed in term of zeta potential. As found by the inventors, (see FIG. 4) a zeta potential of +6 mV does not result in an antiviral effect. Therefore, according to the invention, the nanoparticle of the dimensions indicated above, has also a zeta potential higher than +6 mV, e.g. a zeta potential from 10 to 30 mV.

According to a further embodiment, the nanoparticle of the invention has a zeta potential from 15 to 25 mV, preferably from 18 to 22 mV.

According to the present invention, said nanoparticle and said nanopolymer have nanoscale dimensions of 100-400 nm in all three dimensions and can therefore be defined as a 3 nD (three nanoscale dimensions) nanoparticle or nanopolymer according to Larena et al 2008 J. Phys: Conf. ser 100 102023 pages 1-3 “Classification of nanopolymers”.

In particular, the process of the invention comprises the steps of
a. submitting an amount of positively charged amino acid preferably in dry form, such as in powder form and an amount of Boric Acid preferably in dry form, such as in powder form in an about 1:1 molar ratio to thermal polymerization (alternatively the reagents can be dispersed in water, DMSO or other suitable solvent);
b. purifying the product obtained in step a.

Preferably, the positively charged amino acid is in reagent grade quality or higher, with a purity of at least 98%, preferably ≥98% (TLC). Reagent grade is preferred also for Boric Acid, the purity being also 98% or higher, preferably 99.5% or higher.

According to the invention the positively charged amino acid can be one of L-lysine, known also as (S)-2,6-Diaminocaproic acid, chemical formula

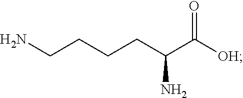

or
L-arginine, known also as (S)-2-Amino-5-guanidinopentanoic acid chemical formula

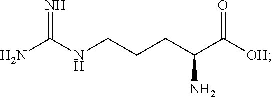 [SMILES: N=C(N)NCCC[C@H](N)C(=O)O]

or
L-histidine, known as (S)-2-Amino-3-(4-imidazolyl)propionic acid chemical formula

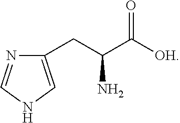 [SMILES: N[C@@H](Cc1c[nH]cn1)C(=O)O]

or one of D-lysine, D-arginine or D-histidine.

In a preferred embodiment, the positively charged amino acid is L-lysine.

Lysine is an amino acid commonly found in protein-rich foods, such as eggs and meat, has been selected as a natural precursor to obtaining highly biocompatible carbon-based nanomaterials. Lysine is a versatile precursor that can form dendrimers and hyperbranched polymeric structures upon thermal polymerization. It can be polymerized to hyperbranched polylysine through polyamidation reactions.

Hyperbranched polymers (HP) are defined as highly branched macromolecules synthesised via a single-step polymerization which have an irregular branching and structure. HP synthesized from amino acids offer several advantages with respect to linear peptides in terms of solubility, biocompatibility, and enhanced proteolytic stability. For these reasons, they are currently under the highlight for developing therapeutic applications.

In general, the formation of hyperbranched lysine polymers without employing any catalyst or protective groups requires several synthesis steps. In the present application a simple approach to produce nanoparticles formed by hyperbranched polylysine (hyperbranched polylysine nanoparticles, HPNs) via a thermal polymerization of a mixture of lysine and H₃BO₃ has been used.

Hence, according to the invention, the thermal polymerisation is carried out by mixing said positively charged amino acid in powder form and said Boric Acid in powder form, heating the mixture thereby obtained at a temperature from 225° C. to 260° for a period of time of 1 to 6 hours and allowing the product resulting from said heating to cool down to 15° C. to 30° C.

Preferably, the thermal polymerisation is carried out by heating the boric acid and positively charged amino acid mixture at a temperature from 230° C. to 250°, even more preferably at a temperature of about 240° C.

The mixture is maintained at the temperature from 225° C. to 260° in the preferred ranges and temperatures defined above for a period of time of 1 to 6 hours, preferably of 1 to 5 hours, more preferably about 5 hours.

The reaction mixture is then allowed to cool down at about room temperature—20° C.

According to the process of the invention, the purification of the product obtained in step a. can be carried out by dispersing the product obtained through said thermal polymerization in a suitable solvent, precipitating the suspension thereby obtained, dialysing the resulting supernatant and freeze drying the dialysis filtrate.

A suitable solvent according to the present invention is milli-Q water, deionized water, DMSO dimethyl sulfoxide.

The dispersing can be carried out by sonication or treatment in ultrasonic bath or any other suitable dispersion treatment. Sonication can be carried out for a period of time of 10 to 20 minutes. In an embodiment of the invention sonication can be carried out for about 15 minutes.

Precipitation can be carried out by centrifugation, preferably at about 8000 to 10000 rpm, even more preferably at about 9000 rpm for a period of time of about 15 to 25 minutes, such as about 20 minutes.

The supernatant is collected and dialysed against water for about 24 hours with a suitable dialysis tube, replacing the water every 12 hours. The filtrate thereby obtained is freeze-dried for a suitable amount of time and then kept at about 4° C.

According to the present invention, the branched nanopolymer obtainable by the process described above and claimed is a hyperbranched nanopolymer. The term hyperbranched nanopolymer is a term that has a well defined meaning in the art and known to the skilled person, see e.g. Sholl et al Journal of polymer science 2007 Vol 45, 5494-5508; Larena et al 2008 J. Phys: Conf. ser 100 102023 pages 1-3 “Classification of nanopolymers”.

The present invention also relates to a nanoparticle comprising a positively charged amino acid branched nanopolymer, wherein said particle has a three-dimensional dimension from 100 to 400 nm in each dimension and a zeta potential of +10-30 mV as defined in the claims. The degree of branching is preferably from 0.25 to 0.5 and can be assessed as shown in the examples.

According to one aspect of the invention, the nanoparticle as defined herein and as claimed is obtainable by the process of the invention, as defined in any of the embodiments above as well as in the claims.

The zeta potential of the nanoparticle of the invention can be assessed in any way known to the skilled person. By way of example, the zeta potential can be assessed by measurement of zeta potential as disclosed in example 8, materials characterization, the hyperbranching can be assessed by ¹H-NMR spectra, the dimensions can be assessed transmission electron microscopy (TEM) and/or by Attenuated total reflectance-Fourier-transform infrared (ATR-FTIR) analysis as disclosed in example 8.

According to the invention, the nanoparticle claimed must have a zeta potential higher than +6 mV, e.g. a zeta potential from 10 to 30 mV.

According to a further embodiment, the nanoparticle of the invention has a zeta potential from 15 to 25 mV, preferably from 18 to 22 mV.

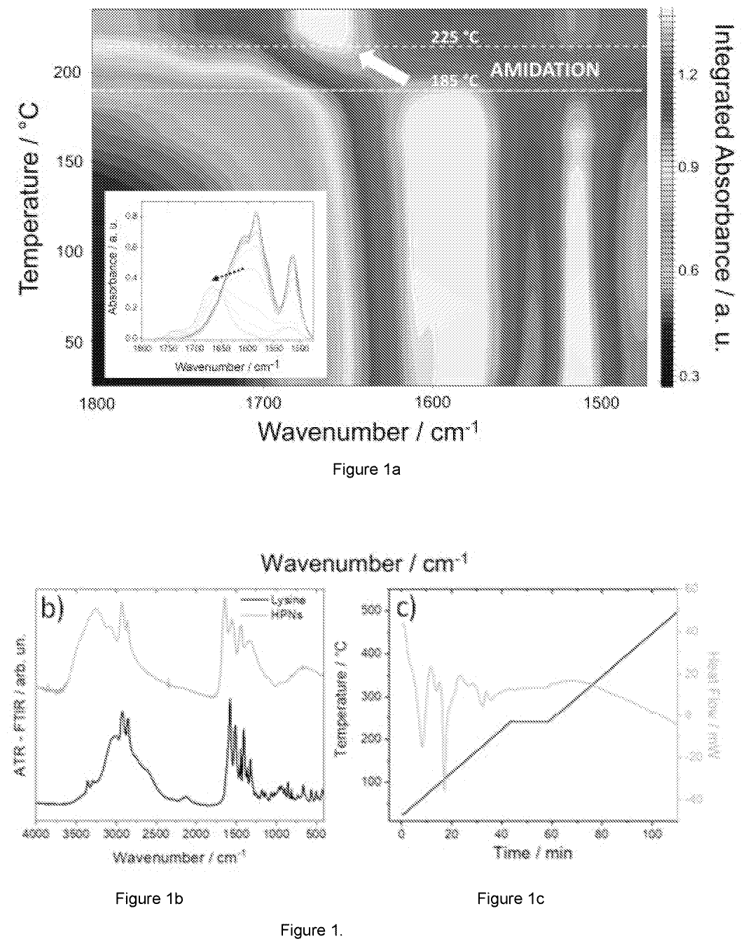

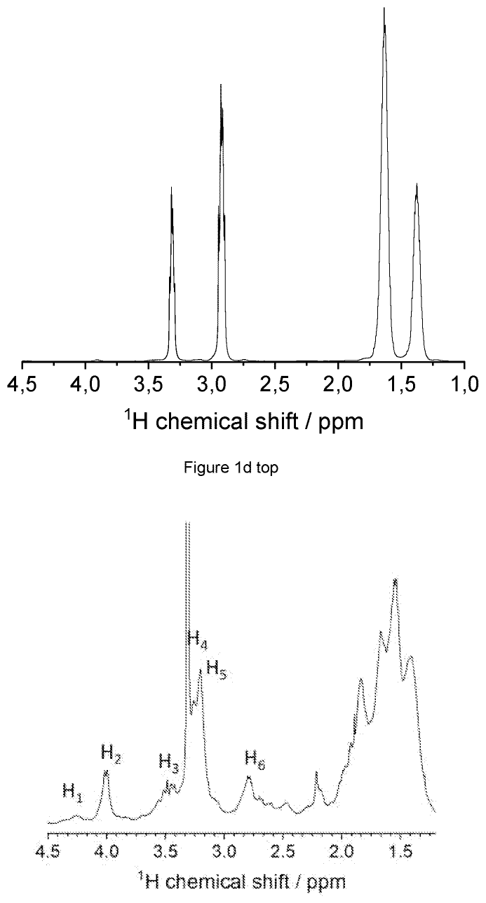

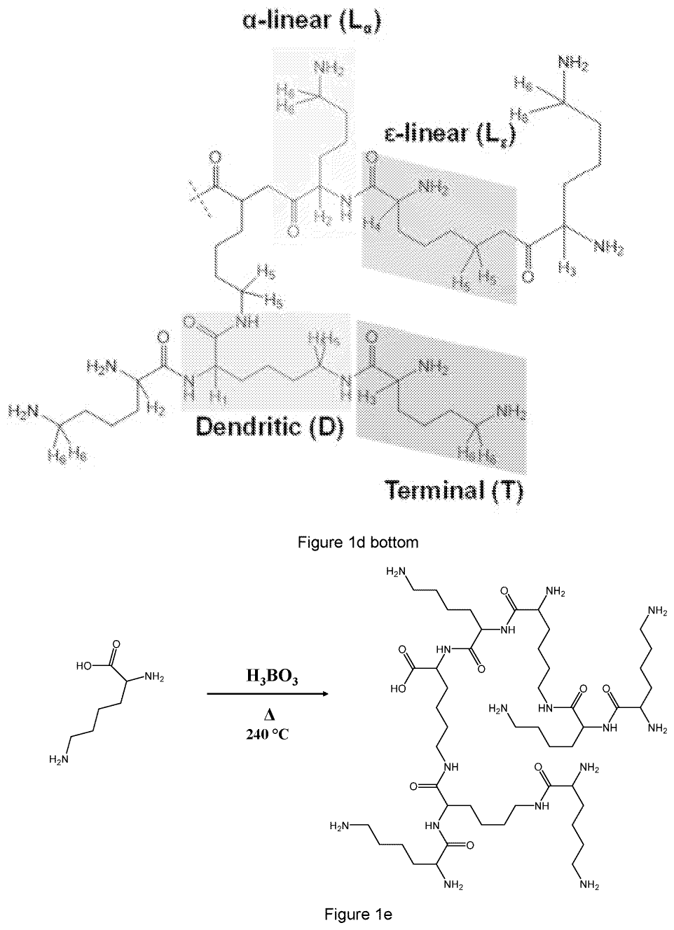

FIGS. 1 and 2 show the characterisation of the lysine-B nanomaterial obtainable by thermal polymerisation of L-lysine with boric acid according to the invention.

According to the invention, the branched nanopolymer obtainable from the method as described above and in the claims is a hyperbranched nanopolymer.

The degree of branched, that can be calculated as exemplified in the examples can be of from 0.25 to 0.5.

In an embodiment of the invention, hence, the nanoparticle of the invention corresponds to the lysine-B nanomaterial obtainable by thermal polymerisation of L-lysine with boric acid according to the invention.

According to said embodiment, said nanoparticle is characterised by the ¹H-NMR spectrum of FIG. 1d.

Still according to said embodiment the nanoparticle is characterised as in FIGS. 1 and 2.

Hence, in a preferred embodiment, the invention relates to a nanoparticle comprising an L-lysine (hyper)branched nanopolymer, wherein said particle has a three-dimensional dimension from 100 to 400 nm in each dimension and a zeta potential of +18-22 mV and a degree of branching of about 0.4.

A further object of the invention is a composition comprising the nanoparticle according to any embodiment described herein and according to the claims and at least one carrier and/or excipient.

Depending on the final form of the composition the skilled person will easily select suitable carrier/s and/or excipient/s.

This composition can be by way of example in the form of a gel, of a solution, of an emulsion. According to an embodiment of the invention, the composition can be a sanitizing or a cleaning composition, and can be loaded onto an adsorbant substrate in order to form a sanitizing wipe or the like. In a further embodiment the sanitizing or cleaning composition can be of food grade quality.

In a preferred embodiment said suitable carrier/s and/or excipient/s are of pharmaceutical grade (i.e. pharmaceutically acceptable) and the composition is a pharmaceutical composition.

The pharmaceutical composition of the invention can be a composition suitable for oral, parenteral, subcutaneous, endovenous, inhalatory, topical, rectal, intravaginal administration.

Hence, accordingly, the nanoparticle or composition as herein defined and claimed can be administered orally, systemically, parenterally, by injection, by endovenous injection, by aerosol, by nebulization, topically, intranasally, nosepharingeally and/or oropharingeally, rectally, intravaginally.

The person skilled in pharmaceutical preparation will readily identify suitable carriers and/or excipients in order to prepare the composition of the invention.

The composition can also be provided already aliquoted in single dosages or in single dosage fractions, or can be provided in a dispenser apt to release single dosage or single dosages fractions, such as, by way of example, a dispenser apt to release a pre-defined amount of a solution in the form of a spray.

A further object of the invention is a product loaded or coated with the nanoparticle or with the composition of the invention in any of the embodiments provided in the present description.

Without being limited thereto, said product can be a sanitizing or cleaning product, a medical device, a medical equipment, an individual protection device, a system for air filtration.

Another object of the invention is the nanoparticle or the composition of the invention in any of the embodiments provided in the present description for use in a medical treatment.

Where allowed the invention encompasses also a medical treatment comprising the administration to a patient in need thereof of the nanoparticle or of the composition of the invention in any of the embodiments provided in the present description in a therapeutically effective amount.

Due to the observed antiviral activity, the nanoparticle or the composition of the invention in any of the embodiments provided in the present description is of particular interest when used in the treatment or prevention, or in adjuvating the treatment or the prevention of viral infections.

The antiviral activity outside and inside the cells demonstrated in the examples below supports the preventive effect as well as the therapeutic effect of the nanoparticles and composition of the invention.

The invention therefore also encompasses a method for the treatment or prevention, or for adjuvating the treatment or the prevention of viral infections or viral diseases wherein therapeutically effective doses of the nanoparticle or of the composition described hereinabove are administered at a therapeutically effective dosage to a subject in need thereof.

The administration can be repeated several times per die and the nanoparticle or the composition can be supplied already in the form of single dosages or with a single-dose dispenser.

Therapeutically effective amount means an amount that is effective in therapy, or an amount sufficient to provide a desired therapeutic effect. An amount that is effective in therapy is an amount which produces one or more desired biological activities during the treatment or the prevention of a disease. In the present case during the treatment or the prevention of a viral infectious disease as defined herein.

According to an embodiment said virus is an animal DNA virus or an animal RNA virus. The viral disease can be an animal disease wherein said animal is a mammal, a fish, a reptile, a bird.

In particular the animal can be a human, a horse, a pig, a bird, a small ruminant (such as goats, sheep and other wild small ruminants, a large ruminant (such as a bovine, an equine, or other wild large ruminants), a rodent, dog, a cat, a primate, a feline, a small or a large bird, chicken, a fish.

According to a preferred embodiment said animal is a human.

By way of example the disease can be African horse sickness (ASH), African swine fever (ASF), Avian influenza (AIV), Bluetongue (BT), Crimean-Congo Haemorrhagic fever (CCHF), Foot and mouth disease (FMD), Newcastle disease (ND), Peste des petits ruminants (PPPR), Porcine Epidemic Diarrhea (PED), Rift Valley fever (RFV), Rinderpest (RPV), Schmallenberg virus (SBV), Schmallenberg virus (SBV), Viral diseases in aquaculture, West Nile Virus (WNV), Canine distemper, Canine influenza, Canine parvovirus, Rabies, Canine Coronavirus gastroenteritis, feline immunodeficiency virus (FIV), Feline Leukaemia Virus (FelV), Feline coronavirus, Feline Infectious Peritonitis (FIP), Feline Parvovirus, Hepatitis, Haemorrhagic fever (Ebola), Encephalitis, Mononucleosis, Covid-19, MERS, Herpesvirus lesions, HIV, Varicella, Gastroenteritis and others.

The conformation and the zeta potential of the nanoparticle of the invention allows said nanoparticle to block potentially any virus provided that the virus has on its surface some negatively charged protein, therefore the virus can be any virus.

A non-limiting example of suitable animal viruses is provided in the table below.

Families of Viruses That Infect Humans

| Family Name | Genome | Capsid Morphology | Envelope |
|---|---|---|---|
| Adenoviridae | dsDNA | Icosahedral | Naked |
| Anelloviridae | ssDNA(−)^{f} | Icosahedral | Naked |
| Arenaviridae | ssRNA(−)^{g}, ^{h} | Helical | Enveloped |
| Astroviridae | ssRNA(+)ⁱ | Icosahedral | Naked |
| Bornaviridae^{a} | ssRNA(−)^{g} | Helical | Enveloped |
| Bunyaviridae | ssRNA(−)^{g}, ^{h} | Helical | Enveloped |
| Caliciviridae | ssRNA(+)ⁱ | Icosahedral | Naked |
| Coronaviridae^{b} | ssRNA(+)ⁱ | Helical | Enveloped |
| Filoviridae^{a} | ssRNA(−)^{g} | Helical | Enveloped |
| Flaviviridae | ssRNA(+)ⁱ | Icosahedral | Enveloped |
| Hepadnaviridae | Partially ssDNA | Icosahedral | Enveloped |
| Hepeviridae | ssRNA(+)ⁱ | Icosahedral | Naked |
| Herpesviridae^{c} | dsDNA | Icosahedral | Enveloped |
| Orthomyxoviridae | ssRNA(−)^{g} | Helical | Enveloped |
| Papillomaviridae | dsDNA | Icosahedral | Naked |
| Paramyxoviridae^{a} | ssRNA(−)^{g} | Helical | Enveloped |
| Parvoviridae | ssDNA | Icosahedral | Naked |
| Picobirnaviridae | dsRNA | Icosahedral | Naked |
| Picobirna | dsRNA | Icosahedral | Naked |
| Picornaviridae^{d} | ssRNA(+)ⁱ | Icosahedral | Naked |
| Pneumoviridae | ssRNA(−)^{g} | Helical | Enveloped |
| Polyomaviridae | dsDNA | Icosahedral | Naked |
| Poxviridae | dsDNA | Complex | Varies |
| Reoviridae | dsRNA | Icosahedral | Naked |
| Retroviridae | ssRNA(+)ⁱ | Complex | Enveloped |
| Rhabdoviridae^{a} | ssRNA(−)^{g} | Helical | Enveloped |
| Togaviridae | ssRNA(+)ⁱ | Icosahedral | Enveloped |
| Delta^{e} | ssRNA(−)^{g} | Icosahedral | Enveloped |

^{a}Mononegavirales.
^{b}Nidovirales.
^{c}Herpesvirales.
^{d}Picornavirales.
^{e}Floating genus, which is not currently assigned to a viral family. It bears some similarities to viroid pathogens of plants.
^{f}ssDNA(+) indicates the mRNA-coding strand.
^{g}ssRNA(−) indicates the complement of the message-sense strand.
^{h}Some segments are ambisense.
ⁱssRNA(+) indicates a message-sense strand.
ds, double stranded; ss, single stranded.

From table 201.2 of “Classification of Human Viruses” R,D, Siegel; Principles and Practice of Pediatric Infectious Diseases. 2018: 1044-1048.e1

In a further embodiment the virus is an animal or human Coronavirus, Orthomyxovirus, Filovirus, Flavivirus, Hepadnavirus, Hepevirus, Herpesvirus, Papillomavirus, Pneumovirus, Poxivirus, Rhinovirus, Reovirus and/or Togavirus.

In a particularly preferred embodiment said Coronavirus is one of SARS, MERS or SARS-CoV-2.

According to the invention, the nanoparticle or composition for can be administered orally, systemically, parenterally, by injection, by endovenous injection, by aerosol, by nebulization, topically, intranasally, nasopharyngeal and/or oropharyngeal, rectally, intravaginally.

All the experiments in the examples below were carried out on the commercially available cell Line Vero E6.

In any part of the present description and claims the term comprising can be substituted by the term “consisting of”.

All the examples were carried out with the nanoparticles comprising a L-Lysine (hyper)branched nanopolymer prepared according to example 7 indicated also as “lysine-B nanomaterial”.

It is herein declared that all experiments involving cells were carried out on commercially available African green monkey Vero E6 cells.

### EXAMPLES

### Example 1—Lysine-Only and Lysine-Based Nanomaterial Structure and Properties

Two distinct carbon nanomaterials, here also indicated as lysine-only nanomaterial or lysine-only nanopolymer when obtained by thermal polymerisation of the L-lysine as sole starting material as opposed to the nanoparticles comprising a L-Lysine (hyper)branched nanopolymer of the invention obtained by via thermal polymerisation of L-Lysine and boric acid (also referred to as lysine-B nanomaterial) were synthesised as described in example 7. The first one employing only pure L-Lysine as the precursor and a second one by using a L-Lysine-H₃BO₃ mixture. The two resulting materials differ for dimension and zeta potential. This difference has been reflected in a much different antiviral response, with lysine-only-nanomaterials which do not exhibit any antiviral activity (see examples and FIG. 4).

One of the differences that appear to be crucial for the antiviral activity of the nanoparticle of the invention is the surface charge, expressed in terms of zeta potential, as indicated above. In fact, while the lysine-B nanomaterial of the invention, e.g. obtainable according to example 7 and characterised in FIG. 1, has a zeta potential of +18-22 mV, the lysine-only nanomaterial has a zeta potential not higher than +6 mV. To understand the effect of the thermal treatment on the derived nanomaterials FTIR in-situ spectroscopy was used to monitor the structural changes as a function of the temperatures. FIG. 1a shows the temperature-wavenumber-intensity infrared graph in the 1800-1450 cm⁻¹ range of the Lysine-B nanomaterial during thermal polymerisation in air from 25 up to 240° C. The FTIR data collected in-situ at increasing temperatures show that the lysine-H₃BO₃ (FIG. 1b) system undergoes to an amidation reaction between 185 and 225° C. in accordance with DSC data (FIG. 1c). The —COO⁻ stretching band at 1580 cm⁻¹ decreases in intensity with the rise of the temperature to transform into the C═O stretching band of amide I peaking around 1654 cm⁻¹. This process gives the formation of polylysine structures via one-step polycondensation of L-lysine in presence of boric acid by the thermal treatment at 250° C. ¹H NMR (FIGS. 1c and 1d) well supports the formation of a hyperbranched polylysine structure in accordance with the literature (FIGS. 1e and 1f). The amidation reactions give polymeric cluster whose dimensions are within the 200-300 nm range when measured by FITR (see FIG. 2d) and of about 150-200 nm when measured with TEM. The final material is also easily dispersed in water and the DMSO solvent used for the in-vitro tests (FIG. 2). FIG. 2a shows the UV-Vis absorption spectra of lysine (black line) and lysine-B nanomaterial (red line) in water. The spectra are characterized by an intense absorption band in the deep UV, around 205 nm, which is assigned to π-π* transitions. At higher wavelengths, a weak absorption band around 270 nm, which involves nitrogen lone pairs, is observed. The lysine-B nanomaterial is fluorescent with an emission in the blue region. FIG. 2b shows the three-dimensional (3D) fluorescence spectra [excitation (y)—emission (x)—intensity (z)] of lysine-B nanomaterial in water with an excitation-dependent emission response. The lysine-B nanomaterial exhibits a broad emission peaked at 450 nm under excitation at 370 nm with a QY efficiency of 4.5%. By comparing the lysine-B nanomaterial optical properties with the lysine precursor, the emission in the blue region appears characteristic of lysine (FIG. 2e left) which has an emission maximum around 440 nm under excitation at 360 nm. The fluorescence of such nanomaterial represents an intrinsic advantage because can be also used at the same time for bioimaging to follow its interactions with the cells.

The TEM images of the lysine-B nanomaterials reveal the formation of quasi spherical amorphous structures (FIG. 2c), in the ≈150-200 nm range. The electronic contrast is very low which does not allow precise identification of the particle boundary. Moreover, the particles appear completely amorphous with no evidence of crystal formation, as observed in different lysine-B nanomaterials and supported by X-ray diffraction analysis (FIG. 2f). These results suggest the formation of interconnected polymeric structures with a low degree of densification. FIG. 2d shows the Dynamic Light Scattering (DLS) measure of the lysine-B nanomaterials in water. The particle distribution is well simulated by a Gaussian curve with a maximum around 300 nm.

Another important characterization of the lysine-B nanomaterial that was performed regards the nature of the zeta potential. The analysis has shown that the zeta potential of the particles is +20±2 mV, in accordance with similar measures done using carbon dots.

### Example 2—Cytotoxicity

Cytotoxicity and antiviral efficacy against SARS-CoV-2 of the lysine-B nanomaterial obtained according to example 7 have been assessed in African green monkey Vero E6 cells. Remdesivir has been used as a reference drug, as it shows antiviral activity in vitro against the new coronavirus and represents the first treatment approved by Food and Drug Administration for COVID-19.

Lysine-B nanomaterial cytotoxicity, defined as the extent to which the nanoparticles disrupt cellular structures or processes related to cell survival and proliferation, has been studied to predict biocompatibility. For the cytotoxicity test, the MTS assay has been applied; it is a widely used methodology employing soluble tetrazolium salts such as 3-(4,5-dimethylthiazol-2-yl)-5-(3-carboxymethoxyphenyl)-2-(4-sulfophenyl)-2H-tetrazolium (MTS), which is reduced by cellular nicotinamide adenine dinucleotide (phosphate)-dependent oxidoreductase enzymes in the presence of an intermediate electron acceptor (phenazine ethosulfate), to form a formazan derivative that is quantified in a spectrophotometer. This product reflects the metabolic activity and thus viability of cells and, hence, can be used to determine a toxic dose of a substance. A standard dedicated to nanomaterials using the MTS assay as an in vitro cytotoxicity assay has been recently published International Standard ISO 19007: 2018(E). Nanotechnologies-In Vitro MTS Assay for Measuring the Cytotoxic Effect of Nanoparticles; ISO: Geneva, Switzerland, 2018. Vero E6 cells have been exposed to increasing concentrations (0.5, 5, 50, 500 μg mL⁻¹) of lysine-B nanomaterial to get the 50% cytotoxic concentration (CC₅₀) value; the data have been compared with the reference drug remdesivir as well as the lysine-based nanomaterial (Table 1).

TABLE 1
Cytotoxicity data (expressed as % viability compared to control,
mean and standard deviation) obtained in MTS assay.

| | | | | Lysine-only nanopolymers | |
|---|---|---|---|---|---|
| Remdesivir | | Lysine-B nanomaterial | | | Cytotoxicity |
| | Cytotoxicity | | Cytotoxicity | | (% cell |
| | (% cell | | (% cell | | viability, |
| | viability, | | viability, | | compared |
| | compared to | | compared to | | to |
| Concentration | untreated | Concentration | untreated | Concentration | untreated |
| (μg mL⁻¹) | control) | (μg mL⁻¹) | control) | (μg mL⁻¹) | control) |
| 300 | TOXIC | 500 | 85.8 ± 2.9 | 125 | 84.5 ± 3.8 |
| 60 | 82.1 ± 2.2 | 50 | 102.5 ± 2.8 | 12.5 | 84.4 ± 7.7 |
| 12 | 98.9 ± 6.7 | 5 | 99.1 ± 1.9 | 1.25 | 86.1 ± 10.2 |
| 2.4 | 94.1 ± 1.0 | 0.5 | 98.8 ± 1.1 | 0.125 | 97.3 ± 0.3 |

500 μg mL⁻¹ was the highest dose that could be tested because of DMSO solvent concentration limits in cell culture. The results show that lysine-B nanomaterial is not cytotoxic up to the maximum tested dose, with a CC₅₀>500 μg mL⁻¹, in accordance to previous biocompatibility tests obtained for carbon dots^{Error! Bookmark not defined.}. Reference drug remdesivir had instead a measurable CC₅₀ value of >120 μg mL⁻¹. 125 μg mL⁻¹ was the highest dose that could be tested for lysine-only nanomaterials because of solubility limits in DMSO. The results show that also the lysine-only nanopolymers are not cytotoxic up to the maximum tested dose, with a CC₅₀>125 μg mL⁻¹.

FIG. 3 shows the cytotoxicity curves of the lysine-B nanomaterial (a) remdesivir (b), and lysine-only nanopolymers (c).

### Example 3—Antiviral Activity

The antiviral assay was performed in parallel with the cytotoxicity experiments, using SARS-CoV-2 permissive cell line Vero E6 from the same culture passage. The cells have been seeded into 96-well plates and exposed, in each assay, to different concentrations of remdesivir, lysine-B nanomaterial obtained according to example 7or lysine-only nanopolymers. Cells have been then infected with SARS-CoV-2 (multiplicity of infection, m.o.i. 0.01) and cultured for 72 hours. At the end of the incubation period, viral replication has been examined through ELISA assay, quantifying SARS-CoV-2 nucleoprotein. Virus-induced cytopathic effect (CPE) resulted in detached cells, as monitored by light microscopy. Antiviral efficacy data (i.e., SARS-CoV-2 nucleocapsid protein, NC, expressed as % of control, mean and standard deviation) obtained in ELISA assay are listed in Table 2.

TABLE 2
Antiviral activity data of test compounds at the different concentrations, i.e.,
% viral nucleocapsid protein, NC, compared to untreated infected control (=100%).

| Remdesivir | | Lysine-B nanomaterial | | Lysine-only nanopolymers | |
|---|---|---|---|---|---|
| | Antiviral | | Antiviral | | Antiviral |
| | activity | | activity | | activity |
| | (% viral | | (% viral | | (% viral |
| | NC | | NC | | NC |
| | protein, | | protein, | | protein, |
| | compared | | compared | | compared |
| | to | | to | | to |
| Concentration | untreated | Concentration | untreated | Concentration | untreated |
| (μg mL⁻¹) | control) | (μg mL⁻¹) | control) | (μg mL⁻¹) | control) |
| 6 | 14 ± 7 | 500 | 14 ± 7 | 125 | 114 ± 20 |
| 1.2 | 73 ± 9 | 50 | 79 ± 41 | 12.5 | 100 ± 0 |
| 0.24 | 96 ± 10 | 5 | 95 ± 19 | 1.25 | 100 ± 0 |
| 0.05 | 107 ± 23 | 0.5 | 94 ± 9 | 0.125 | 100 ± 0 |

The lysine-B nanomaterial was effective in reducing SARS-CoV-2 viral replication, with a 50% inhibitory concentration (IC₅₀) value of 125 μg mL⁻¹. Remdesivir has been used as control of viral infectivity inhibition and had an IC₅₀ of 1.9 μg mL⁻¹, in line with results obtained by other research groups employing different readout methodologies (Wang M. et al Remdesivir and chloroquine effectively inhibit the recently emerged novel coronavirus (2019-nCoV) in vitro. Cell Res. 30, 269-271(2020). On the contrary, lysine-only nanomaterial was not effective in reducing SARS-CoV-2 viral replication, with a 50% inhibitory concentration (IC₅₀) value higher than 125 μg mL⁻¹.

FIG. 4 shows the dose-response curves of the lysine-B nanomaterial (a) remdesivir (b) and lysine only nanopolymers (c). Virus infection positive control has shown marked effects on cell morphology (FIG. 5b) compared to uninfected control (FIG. 5a). lysine-B nanomaterial has shown a degree of protection from cytopathic effect at effective concentrations. The pictures of the cells, taken during treatment with lysine-B nanomaterial at 2×IC₅₀ shown that it was effective in restoring the Vero E6 cell monolayer (FIG. 5c).

### Example 4—Time of Addition

Possible mechanism of action of viral inhibition. Previous reports suggest that SARS-CoV-2 virus binds to the angiotensin converting enzyme 2 (ACE2) receptor with its spike protein. The virus size is around 60-140 nm.

Most of the reported carbon dots play their antiviral activity interfering with the early stage of viral infection by altering the viral surface proteins. Surface-functionalized carbon dots with amine or boronic acid functional groups are effective to inhibit host-cell entry of herpes simplex virus type 1. Curcumin derived carbon dots can block infection at a very early stage of viral entry due to viral aggregation and inactivation caused by electrostatic interaction of positively charged carbon dots. Also, curcumin carbon dots have shown to slow down the production of negative RNA strand in porcine epidemic diarrhea virus. Viral inhibition could also be derived from hindering budding and detachment steps when the progeny of virus is budding from the host cells. Inhibition of another human coronavirus HcoV-229E entry and viral replication was achieved with carbon dots. Huang et al. showed that carbon dots inhibited viral entry of both enveloped (flaviviruses like Zika and dengue viruses) and non-enveloped viruses (e.g. adenovirus-associated virus) by binding directly to viral surface proteins and hinder the first step of viral attachment to the host cell. In order to understand whether similar mechanisms could explain the antiviral activity of lysine-B nanomaterial obtained according to example 7 a time-of-addition experiment was carried out in order to explore which step(s) in the viral life cycle is blocked by said nanomaterials. For the full-time treatment, cells have been pre-treated with lysine-B nanomaterial (a concentration of 4×IC₅₀ was selected for this experiment, i.e. 500 μg mL⁻¹) for 1 h prior to virus infection at 37° C., followed by virus adsorption for 1 h in the presence of lysine-B nanomaterial. Then, cells have been washed and further cultured at 37° C. with the lysine-B nanomaterial-containing medium until the end of the experiment. To examine whether the substance could block viral attachment and entry, a pre-adsorption treatment has been performed, where lysine-B nanomaterial has been added to the cells for 1 h at 37° C. before virus infection and maintained during virus adsorption. Then, the mixture has been replaced with a fresh medium without lysine-B nanomaterial till the end of the experiment. To examine antiviral effect during post-entry steps, such as genome translation and replication, virion assembly and virion release from the cells, a post-adsorption assay has been carried out, in which the lysine-B nanomaterial-containing medium has been added to cells only after virus adsorption and maintained until the experiment's end. The full-time treatment with lysine-B nanomaterial has resulted in a complete viral replication inhibition, measured by quantification of the viral nucleocapsid protein by ELISA assay. The molecule did not show the same degree of protection in the pre-adsorption treatment, as viral replication inhibition was 26% compared to untreated control; in the post-adsorption treatment, inhibition of viral replication was 72% compared to untreated control (FIG. 6). The results confirm that lysine-B nanomaterial may act at different stages of SARS-CoV-2 life cycle. No compound-related cytotoxic effect has been observed on uninfected cells in all experimental settings (not shown in data).

### Example 5—Nanoparticle Uptake Kinetics

In order to confirm whether the lysine-B nanomaterial obtained according to example 7 could penetrate the host cell and therefore have the potential to exert antiviral activity in post-entry steps, they have investigated the nanoparticle uptake into the cells, performing a series of experiments in which Vero E6 cells have been incubated for 6 and 24 hours in growth medium containing effective concentrations of CNPs (2×IC₅₀, 250 μg mL⁻¹, or 4×IC₅₀, 250 μg mL⁻¹). The resulting fluorescence per cell has been evaluated by flow cytometry. Fluorescence intensity overlapped when cells were pulsed with the lysine-B nanomaterial for 6 or 24 hours (FIG. 7a), indicating efficient nanoparticle entry into the cells for short or longer incubation times, compatible with a demonstrated antiviral efficacy. They have also demonstrated that the lysine-B nanomaterial are taken up by the cells in a dose-dependent manner (FIG. 7b, c).

### Example 6—Fluorescence Microscopy

Nanoparticle internalization has been confirmed analyzing Vero E6 cells pulsed with lysine-B nanomaterial for 24 hours by means of confocal microscopy. After 24 h of incubation with 250 μg mL⁻¹ of lysine-B nanomaterial, fluorescent green spots were visible inside the cytoplasm confirming internalization (FIG. 8a, b). Z-stack and 3D rendering have confirmed that the lysine-B nanomaterial is effectively inside the cells and not on the cell surface (FIG. 8c, d). There was no evidence of lysine-B nanomaterial in the cell nuclei (FIG. 8c). The plasma membrane represents a highly selective barrier protecting living cells and limiting entry and exit of large macromolecules. The capability of the lysine-B nanomaterial of the invention of rapidly and effectively penetrate host cells might represent an advantage over drugs with poor permeability properties. Uptake of the nanoparticles by the cellular systems most probably occur with a process known as endocytosis. Further studies will be needed to better elucidate uptake mechanism of lysine-B nanomaterial, to assess whether it is the result of pinocytosis or phagocytosis processes. The SARS-CoV-2 virus is characterized by a spike (S) glycoprotein on the outer surface which plays a critical role in the interaction with antiviral systems and external surfaces. Van der Waals and surface charges are the forces which govern the interaction of the virus through the surface active chemical groups, namely carboxylic acid, hydroxyls, amines and carbonyls. Viral particles, in general, have an isoelectric point below 7 and at neutral pH exhibit a negative charge. Without being bound to theories, the authors suppose that the positive charge of the lysine-B nanomaterial obtained according to example 7 favours the electrostatic interaction with the virus surface and provide a physical wrapping of the virus, whose dimensions are similar, thereby exerting a highly effective antiviral effect. The electrostatic interaction between the positive charged lysine-B nanomaterial and the negative charged virus could explain the antiviral activity, as the nanomaterial could inhibit the viral effectiveness both before and after the entry of the virus into the cells.

The lysine-B nanomaterial prepared according to example 7 has shown an effective antiviral activity against SARS-CoV-2 without being cytotoxic. In addition, the low cost and easy synthesis opens the route for developing new nanomaterials with potential application as antivirals.

### Method Details

### Example 7 Carbon Nanomaterials Preparation

Lysine-only nanopolymers have been synthesized by thermal polymerisation. Commercial L-Lysine powder (Sigma-Aldrich, crystallized, ≥98.0% (NT), H₂N(CH₂)₄CH(NH₂)CO₂H) has been placed in a ceramic crucible and heated up to 240° C. for 5 h and allowed to cool down to 20° C. before further treatment. After the thermal treatment, the obtained brown-black solid has been dispersed in milli-Q water, sonicated for 15 min and then centrifugated at 9000 rpm for 20 min. The supernatant has been collected and dialyzed against water for 24 h with a dialysis tube (benzoylated, avg. flat width 32 mm (1.27 in.), replacing the water every 12 h. The filtrate has been collected and the Lysine-only nanopolymers therein have been freeze-dried for 24 h and kept at 4° C. before characterization.

The nanoparticles comprising a L-Lysine (hyper)branched nanopolymer of the invention, i.e. the nanoparticles characterised in FIGS. 1 and 2, said nanoparticles having a three-dimensional dimension from 100 to 400 nm in each dimension and a zeta potential of +18-22 mV, used in all the tests reported herein, have been prepared as follows: Boric acid powder H₃BO₃, Carlo Erba) and L-Lysine powder (Sigma-Aldrich, crystallized, ≥98.0% (NT), H₂N(CH₂)₄CH(NH₂)CO₂H) have been mixed in a 1:1 molar ratio in a mortar and treated at 240° C. for 5 h. The obtained compound has been sonicated, dialyzed and freeze-dried according to the procedure reported above for Lysine-only nanopolymers. The nanoparticles comprising a L-Lysine (hyper)branched nanopolymer thus obtained have been stored at 4° C. before characterization. The nanoparticles thus obtained are also indicated, in the present description, as lysine-B nanomaterial.

### Example 8 Materials Characterization

Transmission electron microscopy (TEM) bright-field images were obtained by using a FEI TECNAI 200 operating at 200 kV with field emission electron guns. Before analysis, the lysine-B nanomaterial were dispersed in ethanol and ultrasonicated for 10 minutes. Afterward, the solutions containing the lysine-B nanomaterial were cast on grids made by Cu and covered with an ultrathin layer of Carbon (nominally of 3 nm) mounted on a lacey carbon film. After drying at room temperature, the grids were directly used for the measures. The particle size was estimated by measuring at least 10 different particles on 5 images taken from different areas of the grid.

Attenuated total reflectance-Fourier-transform infrared (ATR-FTIR) analysis was carried out by an ATR accessory coupled with an infrared Vertex 70 interferometer (Bruker). The ATR spectra were recorded in the 4000-400 cm⁻¹ range with a 4 cm⁻¹ resolution.

UV-Vis absorption spectra were collected by a Nicolet Evolution 300 UV-Vis spectrophotometer (Thermo Fisher) with a bandwidth of 1.5 nm. In-situ Fourier-transform infrared spectra on sample powders in potassium bromide (KBr, IR 99%, Sigma) were collected in an electrical heating jacket in transmission geometry (Specac).

Fluorescence spectroscopy measurements were performed on a Horiba Jovin Yvon Fluoromax-3. Typically, 3D PL maps of aqueous solutions were recorded from 200 nm to 600 nm. The same spectrofluorometer and identical measurement settings were used in all the cases for simple comparison of the obtained data. Photoluminescence quantum yield (QY) measurements has been performed using the quanta-φ (HORIBA) integrating sphere accessory, attached to the “NanoLog” Horiba Jobin Yvon spectrofluorometer.

X-ray diffraction (XRD) patterns were collected using a SmartLab X-ray powder diffractometer (Rigaku, Tokyo, Japan) in Bragg-Brentano geometry with Cu Kα radiation (λ=1.54178 Å) and a graphite monochromator in the diffracted beam.

¹H-NMR spectra were recorded at 25° C. on a Bruker Avance III 400 MHz spectrometer. Deuterium Oxide (D₂O)+Tetramethylsilane (TMS, 0.05% v/v) was used as the solvent. Deuterium oxide 99.9 atom % D, containing 0.05 wt. % 3-(trimethylsilyl) propionic-2,2,3,3-d4 acid sodium salt was purchased from Sigma-Aldrich. Samples were dissolved in 0.6 ml D₂O and transferred to 5 mm NMR sample tube. TMS, used as internal standard, was calibrated as δ=0.00 ppm. The experimental parameters were: ¹H NMR: Pulse angle of 90°, acquisition time of 2.5 s, 512 repetitions and spectral width of 12 ppm.

The degree of branching (DB) and the average number of branches (ANB) have been calculated using the integrals of the different structural units in the ¹H spectra using the formulas according to Scholl, M., et al Controlling Polymer Architecture in the Thermal

Hyperbranched Polymerization of L-Lysine. Macromolecules 40, 5726-5734 (2007):

$$DB = \frac{D + T}{D + L + T} = \frac{D + T}{D + {L}_{α} + {L}_{ε} + T} \qquad ( 1 )$$

$$and$$

$$ANB = \frac{D}{D + L} \qquad ( 2 )$$

with D the dendritic units, T the terminal structural units, Lₐ the N^{a}-linked linear units, Le the N^{e}-linked linear units (see SI).

Zeta potential (ζ) and hydrodynamic diameter (size) of lysine-B nanomaterial and Lysine-only nanomaterial in solution were measured using a Zetasizer Nano ZSP (Malvern Instruments) in backscatter configuration (θ=173°; laser wavelength of λ=633 nm). The scattering cell temperature was fixed at 298 K, and the data were analyzed through the Zetasizer software 7.03 version. The samples were prepared by dissolving solid samples in milliQ water (1 mg mL⁻¹). Samples were left under rotation for one hour at 25° C. before analysis. All measurements were carried out at least in triplicate.

The zeta potential of the lysine-only nanomaterial obtained according to example 7 was +6 mV, while the zeta potential of the lysine-B nanomaterial obtained according to example 7 was +18-22 mV.

### Viral Isolate

Human 2019-nCoV strain 2019-nCoV/Italy-INMI1 was isolated in Italy (ex-China) from a sample collected on Jan. 29, 2020, from the Istituto Lazzaro Spallanzani, Rome, Italy (ref. available from: https://www.european-virus-archive.com/virus/human-2019-ncov-strain-2019-ncovitaly-inmi1-clade-v).

### Cell Line

Cytotoxicity and antiviral activity of the all the compounds were studied in Vero E6 cells (Cercopithecus aethiops, kidney, ATCC CRL-1586). Cell line was maintained in DMEM supplemented with 1% glutamine, 1% penicillin/streptomycin and 10% fetal bovine serum, FBS (complete medium).

### Cytotoxicity Assay

A cytotoxicity experiment was performed in parallel with the antiviral assay, using cells from the same passage. Exponentially growing Vero E6 cells were seeded into a 96-well plate at 1×10⁵ cells mL⁻¹ in complete medium, 24 hours later cells were exposed to different concentrations of drugs in complete medium (2% FBS, as in antiviral activity assay) for 72 hours. Nanomaterial was resuspended in DMSO and sonicated for 15 minutes. Compound dilutions were performed in culture medium. Remdesivir was included as reference drug.

Cytotoxic effect was evaluated through MTS colorimetric assay (Promega) and confirmed through observation of cell monolayer at the microscope. A cytotoxic concentration 50% (CC₅₀) was calculated through interpolation of the dose-response curves generated by Magellan™ software.

### Antiviral Activity Assay

Exponentially growing Vero E6 cells were seeded into a 96-well plate at their optimal density in complete medium, 24 hours later cells were exposed to different concentrations of drugs. Then cells were infected with SARS-CoV-2 (multiplicity of infection 0.01) and cultured for 72 hours. Two replicates for each concentration point were examined. Two different experiments were performed. At the end of the incubation period, antiviral activity was examined through both ELISA assay (Sino Biological, quantifying SARS-CoV-2 nucleoprotein) as well as through cytopathic effect observation at the microscope. An inhibitory concentration 50% (IC₅₀) value was calculated.

### Time-of-Addition Experiments

Vero E6 cells (1×10⁵ cells mL⁻¹) were seeded into 96-well plates and treated with compound (500 μg mL⁻¹) at different stages of virus infection. For full-time treatment, cells were pre-treated with the compound for 1 h prior to virus infection at 37° C., followed by virus adsorption for 1 h in the presence of the molecule. Then, cells were washed and further cultured at 37° C. with the molecule-containing medium until the end of the experiment. For pre-adsorption treatment, the agent was added to the cells for 1 h at 37° C. before virus infection and maintained during virus adsorption. Then, the mixture was replaced with fresh medium without molecule till the end of the experiment. For post-adsorption assay, the drug-containing medium was added to cells only after virus adsorption and maintained until the end of the experiment. Uninfected cells were included in all experimental settings to exclude possible drug-toxicity CPE. For all the experimental groups, cells were infected with multiplicity of infection 0.01 and absorption was performed for 1 h at 37° C. At the end of the incubation period (72 hours), antiviral activity was examined through ELISA assay.

All conditions were tested in duplicate.

### Flow Cytometry

Vero E6 cells were grown in tissue culture flask 25 cm² until ˜80% confluent, then treated with nanoparticles at 250 μg/mL for 24 hours. Untreated cells were used as control. Then cells were detached and resuspended in ice cold PBS for flow cytometry analysis.

Samples have been acquired on FACSCanto flow cytometer (BD Biosciences) and data analysis was performed using BD FACSDiva software program.

Vero E6 cells were selected, gating out dead cells, and both percentage and mean fluorescent intensity (MFI) of lysine-B nanomaterial treated cells were evaluated (FITC channel). 10000 events were collected for each experiment.

Flow cytometric pictures were generated with FCS Express software.

### Nanoparticle Uptake for Confocal Microscopy

Vero E6 cells were grown in 35 mm coverslips directly into a 6-well plate until ˜60% confluent, then treated with nanoparticles at 250 μg mL⁻¹ for 24 hours. Untreated cells were used as control. Cells were washed in PBS and fixed with paraformaldehyde 4% for 15 minutes, then washed with PBS/1% albumin, then PBS only and finally water. Coverslips were then dehydrated and mounted on slide with 50% glycerol under cover-slip, images were acquired using a Leica TCS SP5 confocal microscopy, with LAS lite 170 image software.

### XPS Analysis

Photoemission data was obtained in a custom designed UHV system equipped with an EA 125 Omicron electron analyser with five channeltrons, working at a base pressure of 10-10 mbar. Core level photoemission spectra (B 1s, C 1s, N 1s and O 1s regions) were collected in normal emission at room temperature with a non-monochromatized Al Kα X-ray source (1486.7 eV) and using 0.1 eV steps, 0.5 s collection time and 20 eV pass energy (FIG. 9).

### Analysis of the C 1s and N 1s Regions

XPS spectra of hyperbranched poly-lysine samples obtained from different lysine/boric acid ratios (Table 1) reveal that there is no trace of boron in the nanopolymer structures after synthesis (Figure XPS a and b). Furthermore, both C 1s and N 1s signals appear to be composed of similar contributions, suggesting that boric acid acts as a catalyst in the formation of the nanopolymers (FIGS. 9c and d).

TABLE 3
Analysis of the C 1s and N 1s regions.

| | C 1s region | | | N 1s region | |
|---|---|---|---|---|---|
| Sample | C—C | C—OH/C—N | O—C═O/N—C═O | C—NH₂ | O═C—NH—C |
| Lysine:Boric | 285.3 eV | 286.4 eV | 288.2 eV | 395.2 eV | 400.4 eV |
| acid | 57.5% | 29.8% | 12.7% | 34.3% | 65.7% |
| 1:0.1 | | | | | |
| Lysine:Boric | 285.2 eV | 286.4 eV | 288.1 eV | 395.2 eV | 400.4 eV |
| acid | 62.1% | 28.7% | 9.2% | 41.6% | 58.4% |
| 1:1 | | | | | |

1. A process for the preparation of a nanoparticle comprising a positively charged amino acid branched nanopolymer, comprising subjecting the positively charged amino acid to thermal polymerization in the presence of boric acid,
wherein said nanoparticle has a three-dimensional dimension from 100 to 400 nm in each dimension and a zeta potential of +10-30 mV.

2. The process of claim 1, wherein the amount of positively charged amino acid and an amount of boric acid in a 1:1 molar ratio to thermal polymerization; and the nanoparticle is purified.

3. The process of claim 1 wherein said positively charged amino acid and said boric acid are in powder form.

4. The process of claim 1 wherein said thermal polymerisation is carried out by mixing said positively charged amino acid in powder form and said boric acid in powder form, heating the mixture thereby obtained at a temperature from 225° C. to 260° C. for a period of time of 1 to 6 hours and allowing the product resulting from said heating to cool down to 15° C. to 30° C.

5. The process of claim 1, wherein said purification is carried out by dispersing the product obtained through said thermal polymerization in a suitable solvent, precipitating the suspension thereby obtained, dialyzing the resulting supernatant and freeze drying the filtrate.

6. The process of claim 1, wherein said branched nanopolymer is a hyperbranched nanopolymer.

7. The process of claim 1, wherein said hyperbranched nanopolymer has a degree of branching of 0.25-0.5.

8. The process of claim 1, wherein said positively charged amino acid is L-Lysine.

9. The process according to claim 8 wherein said zeta potential is +18-22 mV.

10. The process according to claim 8 wherein said degree of branching is 0.4.

11. A nanoparticle comprising a positively charged amino acid branched nanopolymer, wherein said particle has a three-dimensional dimension from 100 to 400 nm in each dimension and a zeta potential of +10-30 mV.

12. The nanoparticle according to claim 11 wherein said branched nanopolymer is a hyperbranched nanopolymer.

13. The nanoparticle according to claim 12 wherein said hyperbranched nanopolymer has a degree of branching of 0.25-0.5.

14. The nanoparticle according to claim 12, wherein said positively charged amino acid is lysine.

15. The nanoparticle according to claim 14 wherein said zeta potential is +18-22 mV.

16. The nanoparticle according to claim 14, wherein said degree of branching is 0.4.

17. A nanoparticle obtainable by the process of claim 1.

18. A composition comprising the nanoparticle of claim 11 and at least one carrier and/or excipient.

19. The composition of claim 18 wherein the at least one carrier and/or excipient are pharmaceutically acceptable.

20. The pharmaceutical composition of claim 19 in a form suitable for oral, parenteral, subcutaneous, endovenous, inhalatory, topical, rectal, intravaginal administration.

21. A product coated with the nanoparticle of claim 11.

22. The product of claim 21 wherein said product is a sanitizing product, a cleaning product, a medical device, a medical equipment, an individual protection device, a system for air filtration.

23. (canceled)

24. A method of treating infection comprising administering to a subject a therapeutically effective amount of the nanoparticle of claim 11 to said subject.

25. The method of claim 24, wherein said infection is caused by an animal DNA virus or an animal RNA virus.

26. The method of claim 24, wherein said subject is a mammal.

27. The method of claim 24, wherein said infection is caused by a Coronavirus, an Orthomyxovirus, a Filovirus, a Flavivirus, an Hepadnavirus, a Hepevirus, a Herpesvirus, a Papillomavirus, a Pneumovirus, a Poxivirus, a Rhinovirus, a Reovirus, a Togavirus, an influenza virus.

28. The method of claim 27, wherein said Coronavirus is SARS, MERS or SARS-CoV-2.

29. The method of claim 24, wherein said nanoparticle er composition is administered orally, systemically, parenterally, by injection, by endovenous injection, by aerosol, by nebulization, topically, intranasally, nosepharyngeally and/or oropharyngeally, rectally, intravaginally.
